(19) United States
(12) Patent Application Publication
(10) Pub. No.: US 2012/0101076 A1
(43) Pub. Date: Apr. 26, 2012

(54) CARBONATE DERIVATIVES FOR THE TREATMENT OF COUGH

(75) Inventors: Riccardo Patacchini, Parma (IT); Pierangelo Geppetti, Parma (IT)

(73) Assignee: CHIESI FARMACEUTICI S.p.A., Parma (IT)

(21) Appl. No.: 13/270,467

(22) Filed: Oct. 11, 2011

(30) Foreign Application Priority Data
Oct. 20, 2010 (EP) 10188152.2

(51) Int. Cl. A61K 31/56; A61P 11/02; A61P 29/00; A61P 11/08; A61M 16/16; A61P 11/10; A61P 11/14; A61M 15/00; A61M 11/04; A61K 31/439; A61P 31/04

(52) U.S. Cl. 514/171; 514/305; 128/203.15; 128/200.23; 128/203.16

(57) ABSTRACT

The invention relates to use of certain quinuclidine carbonate derivatives as cough suppressants, particularly for treating patients with upper respiratory tract infections or asthma.

### CROSS REFERENCES TO RELATED APPLICATIONS

This application is claims priority to European Patent Application No. 10188152.2, filed on Oct. 20, 2010, which is incorporated herein by reference in its entirety.

### BACKGROUND OF THE INVENTION

1. Field of the Invention

The present invention relates to the use of quinuclidine carbonate derivatives for the treatment of cough.

2. Discussion of the Background

Cough (or coughing) is a sudden and often repetitively occurring reflex which helps to clear the large breathing passages of secretions, irritants, foreign particles and bacteria. It can happen voluntarily as well as involuntarily.

Frequent coughing usually indicates the presence of a disease. Many virus and bacteria benefit evolutionarily by causing the host to cough, which helps to spread the disease to new hosts. Most of the time, coughing is caused by a respiratory tract infection but can be triggered by choking, smoking, air pollution, asthma, gastroesophageal reflux disease, post-nasal drip, chronic bronchitis, lung tumors, heart failure, and medications such as angiotensin converting enzyme (ACE) inhibitors.

Guidelines can be found in the medical literature for the categorization of coughing (see Irwin R S and Madison J M., New England Journal of Medicine, 2000, 343(23, 1715-1721) which is incorporated herein by reference in its entirety). A cough which lasts for less than three weeks is generally considered “acute” and viral infections of the upper respiratory tract are the most common cause of an acute cough. A cough which lasts for three to eight weeks duration is categorized as sub-acute, and a cough which exceeds eight weeks is defined as chronic.

Coughing is a common and important respiratory symptom that can produce significant complications and for which many individuals seek medical advice. Dextromethorphan is a drug commonly used as antitussive. However, when taken in excess of the label-specified maximum dosages, it acts as a dissociative hallucinogen. Its mechanism of action is as an NMDA receptor antagonist producing effects similar to those of substances such as ketamine and phencyclidine, and hence several cases of abuse have been reported.

Local application of local anesthetics to airways has been explored to treat coughing. While these agents appear to be effective in preventing reflex bronchoconstriction, they can also induce bronchoconstriction. This paradoxical effect limits the utility of these agents in treating coughing and local airway inflammation, especially in asthmatic patients.

A few studies have investigated the potential effects of anticholinergic agents on coughing. Two clinical trials found ipratropium effective in reducing coughing. In a controlled, double-blind, crossover study (Holmes et al. 1992, Respir. Med., 86:425-429, which is incorporated herein by reference in its entirety), inhaled ipratropium bromide was found to be effective, relative to placebo, in suppressing subjectively described postviral coughing. Ipratropium was also able to diminish citric acid-induced cough in asthmatics in a controlled, double-blind, crossover study (Pounsford et al. 1985, Thorax, 40:662-667, which is incorporated herein by reference in its entirety).

However, ipratropium is endowed with a short duration of action, which is inconvenient for the patient, particularly when seeking relief from nocturnal coughing. The effect of the long acting antimuscarinic tiotropium bromide was also investigated. In Dicpinigaitis et al. (Lung, 2008, 186:369-374, which is incorporated herein by reference in its entirety), said drug, administered once daily (18 μg by inhalation) for 7 days to otherwise healthy adult nonsmokers with acute viral upper respiratory tract infection, turned out to be capable of inhibiting cough reflex sensitivity to inhaled capsaicin. More recently, a study has been presented at the 2009 ATS Annual Meeting showing that tiotropium bromide, when administered intratracheally was able to reduce cough elicited by inhalation of citric acid in ovalbumin-sensitized guinea-pigs (Bouyssou et al., Am. J. Respir. Crit. Care Med., April 2009; 179: A4558, which is incorporated herein by reference in its entirety).

However, long acting anticholinergic drugs such as tiotropium bromide—even when administered by inhalation—may exhibit undesired side effects, in particular cardiac side effects, due to systemic absorption.

Therefore a significant need still exists for more effective and safer antitussive therapy for acute coughs, as well as subacute, and chronic coughs. In particular, it would be highly advantageous to provide anticholinergic drugs being highly effective as antitussive agents and having a long duration of action upon inhalation, but, once adsorbed, degraded to inactive compounds which are devoid of any systemic side effects typical of muscarinic antagonists.

WO 2009/090088, which is incorporated herein by reference in its entirety, discloses quinuclidine carbonate derivatives which are consistently and rapidly transformed into inactive metabolites after passing into human plasma.

### SUMMARY OF THE INVENTION

Accordingly, it is one object of the present invention to provide novel methods for treating cough.

It is another object of the present invention to provide novel methods for treating an acute cough.

It is another object of the present invention to provide novel methods for treating a sub-acute cough.

It is another object of the present invention to provide novel methods for treating a chronic cough.

These and other objects, which will become apparent during the following detailed description, have been achieved by the inventors' discovery that compounds of general formula (I):

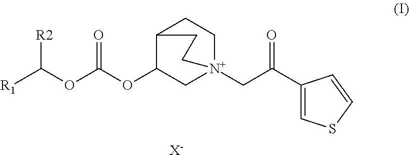

wherein:

R₁ and R₂ are the same or different and are independently selected from the group consisting of H, (C₃-C₈)-cycloalkyl, aryl, and heteroaryl, wherein said aryl or heteroaryl may be optionally substituted with a halogen atom or with one or more substituents independently selected from the group consisting of —OH, —O—(C₁-C₁₀)-alkyl, oxo (═O), —SH, S—(C₁-C₁₀)-alkyl, —NO₂, —CN, —CONH₂, COOH, —(C₁-C₁₀)-alkoxycarbonyl, —(C₁-C₁₀)-alkylsulfanyl, —(C₁-C₁₀)-alkylsulfinyl, —(C₁-C₁₀)-alkylsulfonyl, —(C₁-C₁₀)-alkyl, and —(C₁-C₁₀)-alkoxyl, or

when R₁ and R₂ are both independently aryl or heteroaryl they may be linked through a Y group which may be a —(CH₂)ₙ— group (where n=0, 1 or 2), wherein when n=0, Y is a single bond, forming a tricyclic ring system wherein any of the carbon atoms of —(CH₂)ₙ— may be substituted by a heteroatom selected from O, S, and N, and with the proviso that R₁ and R₂ are never both H; and

X⁻ is a pharmaceutically acceptable anion,

are useful in the treatment of cough.

Thus, in a first embodiment, the present invention provides a method for treating cough, comprising administering an effective amount of a compound according to formula (I):

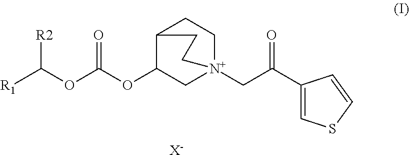

wherein:

R₁ and R₂ are the same or different and are independently selected from the group consisting of H, (C₃-C₈)-cycloalkyl, aryl, and heteroaryl, wherein said aryl or heteroaryl may be optionally substituted with a halogen atom or with one or more substituents independently selected from the group consisting of —OH, —O—(C₁-C₁₀)-alkyl, oxo (═O), —SH, —S—(C₁-C₁₀)-alkyl, —NO₂, —CN, —CONH₂, —COOH, —(C₁-C₁₀)-alkoxycarbonyl, —(C₁-C₁₀)-alkylsulfanyl, —(C₁-C₁₀)-alkylsulfinyl, —(C₁-C₁₀)-alkylsulfonyl, —(C₁-C₁₀)-alkyl, and —(C₁-C₁₀)-alkoxyl, or

when R₁ and R₂ are both independently aryl or heteroaryl they may be linked through a Y group which may be a —(CH₂)ₙ— group (where n=0, 1 or 2), wherein when n=0, Y is a single bond, forming a tricyclic ring system wherein any of the carbon atoms of —(CH₂)ₙ— may be substituted by a heteroatom selected from O, S, and N, and

with the proviso that R₁ and R₂ are never both H; and

X⁻ is a pharmaceutically acceptable anion,

to a subject in need thereof.

According to another aspect, the present invention provides kits-of-parts comprising for separate, sequential or simultaneous administration, a compound of formula (I) and a second therapeutic substance selected from the group consisting of cough suppressants (antitussives), antihistamines, expectorants, decongestants, analgesics, antipyretics, antibiotics, local anesthetics, corticosteroids, and bronchodilators; and one or more pharmaceutically acceptable excipients.

According to a further aspect, the present invention provides pharmaceutical compositions comprising a compound of formula (I) and, optionally, a second therapeutic substance selected from the group consisting of cough suppressants (antitussives), antihistamines, expectorants, decongestants, analgesics, antipyretics, antibiotics, local anesthetics, corticosteroids, and bronchodilators; and one or more pharmaceutically acceptable excipients.

According to yet another aspect, the present invention provides inhalers or nasal spray devices comprising a pharmaceutical composition of the present invention.

### DETAILED DESCRIPTION OF THE PREFERRED EMBODIMENTS

The citric acid cough challenge is a well-established and validated protocol for the assessment of cough suppression. The present inventors tested one of the compounds of formula (I) in this challenge test, in both normal guinea pigs and sensitized guinea pigs. It was surprisingly discovered that that compound performs even better in terms of cough suppression than tiotropium bromide, an anticholinergic that had previously been proposed as a promising antitussive agent (see Dicpinigaitis et al., supra).

A particularly large and significant suppression of coughing was achieved when sensitized guinea pigs were treated with the compound of formula (I). Sensitized guinea pigs closely mimic the human asthmatic state, including airway hyperresponsiveness (AHR). Therefore the compounds of formula (I) show great promise in treating and relieving cough symptoms of allergic asthma.

These results, coupled with the knowledge that compounds of formula (I) have little or no systemic pharmacological activity suggest that such compounds can be employed as efficacious and safe antitussive agents.

In a preferred embodiment, groups R₁ and R₂ of the compound of formula (I) are each aryl or heteroaryl, and are each preferably substituted with a halogen atom. In a particularly preferred embodiment, R₁ and R₂ are the same, and are each aryl with a fluorine substituent.

The term “halogen atom” includes fluorine, chlorine, bromine, and iodine.

The expression “(C₃-C₈)-cycloalkyl” refers to cyclic non-aromatic isolated hydrocarbon saturated groups. Examples include cyclopropyl, cyclobutyl, cyclopentyl, cyclohexyl, cycloheptyl, and cyclooctenyl.

The expression “aryl” refers to mono-, bi-, or tricyclic ring systems having 5 to 20, preferably from 5 to 15, ring atoms, and wherein at least one ring is aromatic. Optionally, one or more hydrogen atoms in said rings can be replaced by one or more halogen atoms or phenyl.

The expression “heteroaryl” refers to mono-, bi-, or tricyclic ring systems having 5 to 20, preferably from 5 to 15, ring atoms, in which at least one ring is aromatic and in which at least one ring atom is a heteroatom (e.g., N, S, or O). Optionally, one or more hydrogen atoms in said rings can be replaced by one or more halogen atoms.

The physiologically acceptable anion (X⁻) of the pharmaceutically acceptable salts used in the invention can be selected by the skilled person. This anion is optionally chloride, bromide, iodide, sulfate, phosphate, methanesulfonate, nitrate, maleate, acetate, citrate, fumarate, tartrate, oxalate, succinate, benzoate. p-toluenesulfonate, preferably chloride, bromide or iodide, more preferably chloride or bromide, and most preferably chloride.

In a particularly preferred embodiment, the invention uses the following compound, of formula (Ia):

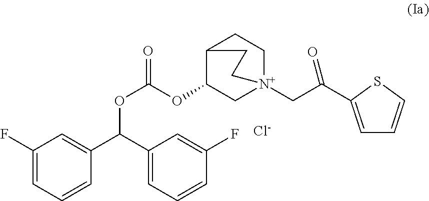

The compounds of formula (I) can be synthesized by any convenient route, for instance as disclosed in WO 2009/090088, which is incorporated herein by reference in its entirety. Example 14 of WO 2009/090088 describes the synthesis of the compound of formula (Ia).

The compounds of formula (I) may be used in substantially pure (R) or (S) enantiomeric forms, or in a mixture of enantiomers in any desired enantiomeric ratio.

The compounds of formula I can be administered, for instance, at a dosage of 0.001 to 500 mg/day, preferably 0.1 to 1 mg/day. The precise dosage for optimal clinical benefit can be determined by a skilled professional in the field.

The compounds of the invention can be administered to a patient in combination with a second therapeutic substance (e.g., any other OTC drug or prescription medicine) used to treat the causes or symptoms of cough or other symptoms of upper respiratory tract infections (URTIs), such as other cough suppressants (antitussives; e.g., dextromethorphan, codeine, dihydrocodeine, hydrocodone, clobutinol, chlophendianol, pentoxyverine, benzonatate), antihistamines (e.g., brompheniramine, chlorpheniramine, desloratidine, dexbrompheniramine, diphenhydramine, promethazine, triprolidine, promethazine), expectorants (e.g., guaifenesin), decongestants (e.g., pseudoephedrine, phenylephrine), analgesics/antipyretics (e.g., acetaminophen, NSAIDs), antibiotics, local anesthetics (e.g., proparacaine, procaine, tetracaine, hexylcaine, bupivacaine, lidocaine, benoxinate, mepivacaine, prilocalne, mexiletene, vadocaine, etidocaine), corticosteroids, or bronchodilators.

The present invention in another aspect relates to a kit-of-parts comprising, for separate, sequential or simultaneous administration, a compound of the present invention and a second therapeutic substance selected from the group consisting of: cough suppressants (antitussives), antihistamines, expectorants, decongestants, analgesics, antipyretics, antibiotics, local anesthetics, corticosteroids, and bronchodilators; and one or more pharmaceutically acceptable excipients. Alternatively, the second therapeutic substance can be a substance obtained or extracted from a natural source (e.g., Echinacea, tea tree oil, turmeric, menthol) or any other substance alleged to promote recovery from respiratory infections or relieve their symptoms (e.g. zinc, vitamin C).

In accordance with the present invention, the compounds of formula (I) may be administered to a patient by any convenient means, such as by pulmonary, oral, nasal, or local administration. Preferably, they are administered by inhalation.

In accordance with the present invention, the compounds of formula (I) may be administered to a patient in any suitable dosage form. Suitable dosage forms include: solutions, suspensions, dry powders, syrups, sprays, gels, drops, aerosols, tablets, elixirs, injections, capsules, and lozenges. Optionally, the dosage form comprises an extended release formulation of the compound.

Pharmaceutical compositions can be prepared comprising a compound of formula (I) formulated together with one or more pharmaceutically acceptable excipients. Suitable pharmaceutical excipients depend on the dosage form and can be selected by the skilled person (e.g., by reference to the Handbook of Pharmaceutical Excipients, 6^{th} Edition, 2009, eds. Rowe et al).

For instance, solid dosage forms may comprise pharmaceutically acceptable excipients such as diluents, suspending agents, solubilizers, buffering agents, binders, lubricants, glidants, coatings, disintegrants, preservatives, colorants, flavorants, lubricants, and the like.

Liquid dosage forms may comprise pharmaceutically acceptable excipients such as diluents, preservatives, wetting agents, sweeteners, flavorants, emulsifiers, suspending agents, and the like.

Inhalable preparations include inhalable powders (dry powders), propellant-containing metering aerosols, and propellant-free inhalable formulations. Dry powders are typically stored in a foil “blister” of a blister pack or in a single dose capsule. Inhalation aerosols comprising a propellant gas such as hydrofluoroalkanes may comprise the compounds of the invention either in solution or in dispersed form. Propellant-driven formulations may also comprise other ingredients such as co-solvents, stabilizers etc. Typically, an aerosol canister for use in an inhaler device will contain multiple doses of the formulation, although it is possible to have single dose canisters as well. Propellant-free inhalable formulations may be in the form of solutions or suspensions in an aqueous, alcoholic, or hydroalcoholic medium.

A nasal spray composition in powder form may comprise a suitable powder base such as talc, lactose starch, or the like. A nasal spray composition in droplet or spray form may comprise an aqueous carrier, e.g., a saline solution comprising about 0.1% to about 2.0% by weight of a salt, e.g., sodium chloride. The nasal composition can be isotonic, i.e., having the same osmotic pressure as blood and lacrimal fluid.

Optionally, the pharmaceutical composition and combinations useful in practicing the present invention are provided to the patient in the form of devices adapted for inhalation or nasal spray. Suitable devices include pressurized meter dose inhalers (pMDIs), breath activated inhalers (MDIs or dry powder inhalers), inhaler devices with spacers, nebulisers (e.g., jet, ultrasonic, or soft-mist nebulizers), intranasal pump dispensers, and squeeze bottles.

Thus, in another aspect, the present invention provides an inhalation or nasal spray device (or an integral component thereof, such as an aerosol canister or a capsule) comprising a pharmaceutical composition comprising a compound of general formula I, and optionally a second therapeutic substance, and one or more pharmaceutically acceptable excipients.

The types of coughs treatable using the method of the invention may be acute, sub-acute, or chronic. An “acute cough” means a cough lasting <3 weeks. A “sub-acute cough” lasts 3-8 weeks. A “chronic cough” means a cough lasting >8 weeks.

In one embodiment the present invention relates to the suppression of an acute or sub-acute cough. Acute cough is commonly associated with an upper respiratory tract infection (URTI). Other causes of acute coughs include: acute bacterial sinusitis, pertussis, exacerbations of COPD, allergic rhinitis, environmental irritant rhinitis, asthma, congestive heart failure, pneumonia, aspiration syndromes, and pulmonary embolism.

In an alternative embodiment, the present invention relates to suppression of a chronic cough, such as coughs associated with emphysema, chronic bronchitis, asthma, gastrooesophageal reflux, post-nasal drip, and post-infectious coughs.

In a preferred embodiment, the present invention relates to suppression of cough associated with asthma.

In another embodiment, the present invention relates to suppression of a cough caused by administration of another medicament, in particular an ACE inhibitor or any medicament used to treat asthma or COPD that tends to provoke a cough response.

“Treatment” of cough (or a cough or coughing) or “suppression” of cough (or a cough or coughing) means reducing the frequency of cough events and/or reducing the severity of the cough events (relative to the non-treated condition). These terms refer to both treatment by prevention and treatment/suppression of cough episodes.

The compounds of the present invention may be administered to a patient at a fixed frequency as prescribed by a doctor, for instance in single or multiple doses, typically once, twice or several times daily. Alternatively, the compounds of the present invention can be administered by a caregiver or self-administered by the patient on an as-needed (pro re nata) basis, in response to symptoms.

In one embodiment, the present invention relates to a method for suppressing a cough comprising administration to a patient in need thereof a therapeutically effective amount of a compound of general formula (I).

A “therapeutically effective amount” of a substance refers to an amount which leads to a clinically significant reduction in the frequency or severity of cough events.

Other features of the invention will become apparent in the course of the following descriptions of exemplary embodiments which are given for illustration of the invention and are not intended to be limiting thereof.

### EXAMPLES

Evaluation of the activities of Compound Ia and tiotropium bromide in an animal cough challenge model.

### Animals.

Male Dunkin-Hartley guinea pigs (250-350 g, Charles-River, Italy) were acclimatized in cages, (24±0.5° C.) for 1 week after delivery, with free access to water and standard rodent diet. One group of guinea pigs was actively sensitized by an intra-peritoneal injection of ovalbumin (100 mg/kg) followed by a subcutaneous injection of ovalbumin (100 mg/kg). Controls received the vehicle alone (0.9% NaCl).

### Experimental Set-Up.

After the period of acclimatization to laboratory conditions, animals were individually placed in a transparent perspex box (20×10×10 cm, Vetrotecnica, Italy) ventilated with a constant airflow of 400 ml/minute. A tussive agent (citric acid, 0.25 M) was nebulized via a mini-ultrasonic nebulizer (Ugo Basile, Italy). The particle sizes produced had an aerodynamic mass median diameter of 0.9 μm, and the output of the nebulizer was 0.4 ml per minute. The numbers of elicited cough efforts were counted by a blind observer.

### Study Protocols.

All experiments were carried out at the same time of day, starting at 9.00 a.m. Guinea-pigs received Compound Ia (1 mM) or tiotropium bromide (0.3 mM) or their vehicle (distilled water) for 10 minutes by aerosol at least 3 hour before the citric acid challenge (0.25 M; for 10 minute; by aerosol), in order to elicit cough.

### Data Analysis.

Values are presented as mean±SEM. Comparisons among groups were made by one way analysis of variance (ANOVA) and the Student's t-test or the Bonferroni's test when appropriate. A p value of <0.05 was considered significant.

### Results.

Compound Ia pretreatment (1 mM; by aerosol, 3 hours before) significantly reduced the number of cough efforts induced by citric acid (0.25 M; by aerosol). The percentage (%) reduction of number of coughs produced by 1 mM Compound Ia was 37.2±5.9% in ovalbumin-sensitized animals and 17.4±6.4% in control, non-sensitized, animals.

Tiotropium bromide (0.3 mM; by aerosol, 3 hours before) showed a tendency to reduce the number of cough efforts induced by citric acid. The percentage (%) of reduction induced by pretreatment with tiotropium bromide was 28.1±11% in ovalbumin-sensitized animals and 20.2±9.9% in control animals. In both animal groups, the effects of tiotropium did not reach a statistical significance.

### CONCLUSIONS

The data show that Compound Ia at a dosage (1 mM) previously shown to produce an antibronchoconstrictor effect in guinea-pigs significantly reduces coughs elicited by citric acid, and that the effect of Compound Ia is more pronounced in ovalbumin-sensitized animals than in control animals. This shows that Compound Ia has the ability to significantly reduce coughing in an asthmatic context. The effect of Compound Ia is mimicked by tiotropium at a dosage (0.3 mM) previously shown to produce antibronchoconstrictor effects comparable to Compound Ia (1 mM). However, the effects produced by tiotropium did not reach a statistical significance. Notably, the effects of Compound Ia administered by inhalation at a “therapeutic-like dosage” exerts antitussive effects that are quantitatively comparable to the effect produced by dextromethorphan (30 mg/kg, i.p) against the same stimulus (Geppetti et al., unpublished).

Therefore, the compounds of formula (I) (including compound (Ia)) are candidates for development of novel antitussive treatments with optimal safety and efficacy profiles, presenting advantages relative to the preferred cough suppressants in current clinical use.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for the treatment of cough, comprising administering an effective amount of a compound according to formula (I): 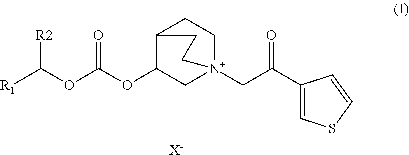
wherein
R₁ and R₂ are the same or different and are independently selected from the group consisting of H, (C₃-C₈)-cycloalkyl, aryl, and heteroaryl, wherein said aryl or heteroaryl may be optionally substituted with a halogen atom or with one or more substituents independently selected from the group consisting of —OH, —O—(C₁-C₁₀)-alkyl, oxo (═O), —SH, —S—(C₁-C₁₀)-alkyl, —NO₂, —CN, —CONH₂, —COOH, —(C₁-C₁₀)-alkoxycarbonyl, —(C₁-C₁₀)-alkylsulfanyl, —(C₁-C₁₀)-alkylsulfinyl, —(C₁-C₁₀)-alkylsulfonyl, —(C₁-C₁₀)-alkyl, and —(C₁-C₁₀)-alkoxyl, or
when R₁ and R₂ are both independently aryl or heteroaryl they may be linked through a Y group which may be a —(CH₂)ₙ— group (where n=0, 1 or 2), wherein when n=0, Y is a single bond, to form a tricyclic ring system wherein any of the carbon atoms of —(CH₂)ₙ— may be substituted by a heteroatom selected from O, S, and N, and
with the proviso that R₁ and R₂ are never both H; and
X⁻ is a pharmaceutically acceptable anion,
to a subject in need thereof.

2. A method according to claim 1, wherein R₁ and R₂ are independently aryl or heteroaryl, each optionally substituted with a halogen atom.

3. A method according to claim 2, comprising administering an effective amount of the compound of formula (Ia): 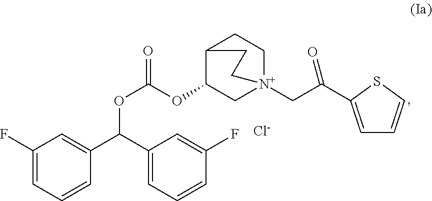
to said subject in need thereof.

4. A method according to claim 1, wherein said coughing is an acute cough.

5. A method according to claim 1, wherein said coughing is a cough associated with an upper respiratory tract infection.

6. A method according to claim 1, wherein said coughing is a chronic cough.

7. A method according to claim 6, wherein said coughing is associated with allergic asthma.

8. A method according to claim 1, wherein said compound of formula (I) is administered in a pharmaceutical composition comprising a compound of formula (I), and one or more pharmaceutically acceptable excipients.

9. A method according to claim 1, wherein said compound of formula (I) is administered in a pharmaceutical composition comprising:
a compound of formula I;
a second therapeutic substance selected from the group consisting of a cough suppressant, an antihistamine, an expectorant, a decongestant, an analgesic, an antipyretic, an antibiotic, a local anesthetic, a corticosteroids, and a bronchodilators; and
one or more pharmaceutically acceptable excipients.

10. A method according to claim 8, wherein said pharmaceutical composition is administered with an inhalation or nasal spray device.

11. A method according to claim 9, wherein said pharmaceutical composition is administered with an inhalation or nasal spray device.

12. A kit, comprising, for separate, sequential or simultaneous administration:
a compound of formula (I): 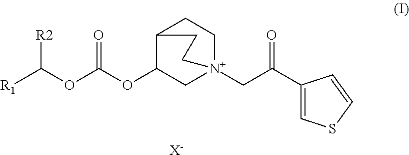
wherein
R₁ and R₂ are the same or different and are independently selected from the group consisting of H, (C₃-C₈)-cycloalkyl, aryl, and heteroaryl, wherein said aryl or heteroaryl may be optionally substituted with a halogen atom or with one or more substituents independently selected from the group consisting of —OH, —O—(C₁-C₁₀)-alkyl, oxo (═O), —SH, —S—(C₁-C₁₀)-alkyl, —NO₂, —CN, —CONH₂, —COOH, —(C₁-C₁₀)-alkoxycarbonyl, —(C₁-C₁₀)-alkylsulfanyl, —(C₁-C₁₀)-alkylsulfinyl, —(C₁-C₁₀)-alkylsulfonyl, —(C₁-C₁₀)-alkyl, and —(C₁-C₁₀)-alkoxyl, or
when R₁ and R₂ are both independently aryl or heteroaryl they may be linked through a Y group which may be a —(CH₂)ₙ— group (where n=0, 1 or 2), wherein when n=0, Y is a single bond, to form a tricyclic ring system wherein any of the carbon atoms of —(CH₂)ₙ— may be substituted by a heteroatom selected from O, S, and N, and
with the proviso that R₁ and R₂ are never both H; and
X⁻ is a pharmaceutically acceptable anion;
a second therapeutic substance selected from the group consisting of a cough suppressant, an antihistamine, an expectorant, a decongestant, an analgesic, an antipyretic, an antibiotic, a local anesthetic, a corticosteroid, and a bronchodilator; and
one or more pharmaceutically acceptable excipients.

13. An inhalation or nasal spray device or integral component thereof, comprising:
a compound of formula (I): 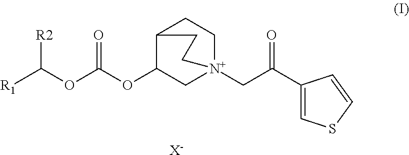
wherein
R₁ and R₂ are the same or different and are independently selected from the group consisting of H, (C₃-C₈)-cycloalkyl, aryl, and heteroaryl, wherein said aryl or heteroaryl may be optionally substituted with a halogen atom or with one or more substituents independently selected from the group consisting of —OH, —O—(C₁-C₁₀)-alkyl, oxo (═O), —SH, —S—(C₁-C₁₀)-alkyl, —NO₂, —CN, —CONH₂, —COOH, —(C₁-C₁₀)-alkoxycarbonyl, —(C₁-C₁₀)-alkylsulfanyl, —(C₁-C₁₀)-alkylsulfinyl, —(C₁-C₁₀)-alkylsulfonyl, —(C₁-C₁₀)-alkyl, and —(C₁-C₁₀)-alkoxyl, or
when R₁ and R₂ are both independently aryl or heteroaryl they may be linked through a Y group which may be a —(CH₂)ₙ— group (where n=0, 1 or 2), wherein when n=0, Y is a single bond, to form a tricyclic ring system wherein any of the carbon atoms of —(CH₂)ₙ— may be substituted by a heteroatom selected from O, S, and N, and
with the proviso that R₁ and R₂ are never both H; and
X is a pharmaceutically acceptable anion;
a second therapeutic substance selected from the group consisting of a cough suppressant, an antihistamine, an expectorant, a decongestant, an analgesic, an antipyretic, an antibiotic, a local anesthetic, a corticosteroid, and a bronchodilator; and
one or more pharmaceutically acceptable excipients.

14. A device or integral component thereof according to claim 11, which is a single- or multi-dose dry powder inhaler, a metered dose inhaler, or a nebulizer.
